(19) United States
(12) Patent Application Publication
(10) Pub. No.: US 2026/0335181 A1
(43) Pub. Date: Oct. 29, 2026

(54) IMPROVEMENTS RELATING TO RECIPROCATING DEVICES

(71) Applicant: Performance Industries AS, Oslo (NO)

(72) Inventor: Espen JENSEN, KRISTIANSAND (NO)

(73) Assignee: Performance Industries AS, Oslo (NO)

(21) Appl. No.: 19/117,870

(22) PCT Filed: Oct. 3, 2023
(86) PCT No.: PCT/NO2023/060061

§ 371 Date: Apr. 2, 2025

(30) Foreign Application Priority Data
Oct. 5, 2022 (NO) 20221062

(51) Int. Cl. A61H 23/02; A61H 9/00; A61H 23/00

(52) U.S. Cl. CPC A61H 23/0254; A61H 9/0057; A61H 23/006; A61H 2023/002; A61H 2201/0153; A61H 2201/0192; A61H 2201/1215; A61H 2201/1436; A61H 2201/149; A61H 2201/1664; A61H 2201/1685; A61H 2201/5023

(57) ABSTRACT

Reciprocator devices for acting upon a region or part of a human or animal body for producing at least one effect thereupon are exemplified, having: a device body; an adjustable drive mechanism; a movable part coupled to the drive mechanism, the drive mechanism being configured to move the movable part reciprocally back and forth with respect to the device body; the drive mechanism being user-adjustable to vary the obtainable range of travel of the movable part. The reciprocator device is a massage gun in one example. Reciprocator devices are also described which in certain examples may be used for vacuum therapy and include a sealable structure and a movable part that is movable back and forth, to produce alternating pull/push forces upon the area of the body enclosed by the structure, and where the movable part is lockably coupled to the enclosed area through a vacuum lock effect.

### CROSS-REFERENCE TO RELATED APPLICATIONS

This application is the U.S. national stage application of International Application No. PCT/NO2023/060061, filed Oct. 3, 2023, which international application was published on Apr. 11, 2024, as WO 2024/076244 in the English language. The International Application claims priority to Norwegian Patent Application No. 20221062, filed Oct. 5, 2022. The international application and Norwegian application are both incorporated herein by reference, in their entirety.

### FIELD

The present invention relates to reciprocating devices, and in particular to reciprocating devices for use on the body of a human or an animal, in particular for purposes of: stimulating or activating blood circulation; stimulating or activating neurons; stimulating, activating, exercising, or massaging muscles and/or other tissue; improving performance, recovery or function of the body or bodily processes; activating or stimulating male reproductive muscles and/or parts; therapy; medical or cosmetic treatment; anti-aging; and/or cellulite reduction.

### BACKGROUND

Various reciprocating devices for use on the human or animal body are known in the prior art, amongst these for example hand-held devices or products such as massage guns.

Such a reciprocating device generally has a component that is movable reciprocally back and forth and means for generating the reciprocal movement of the component. In the case of a massage gun for example, the component can be a massage head, and the movement may comprise vibrations of the massage head for “vibration therapy” or larger reciprocating movements, for example typically 12 mm of travel back or forth, for “percussive therapy”.

The inventors have appreciated that reciprocating devices in use today, particularly in smaller-scale products, may have limited effect when applied to a particular body. For example, body shapes and sizes vary, and a reciprocating device with certain performance characteristics providing a good effect for one person and/or muscle group may not be suitable for others.

Furthermore, the physical condition of individuals can vary. Medical disorders, aging, and/or other environmental and/or biological capacities may limit and/or reduce the performance or function of the body and/or its processes. The obtainable effect or response of one person may be quite different from that of another. The inventors appreciate that good arterial blood flow with high levels of oxygen and nutrients, well-functioning muscles and neurons can play an essential role in the performance of bodily functions and purposes.

The inventors see a need therefore for improved reciprocating solutions, in particular that can be more efficient for different individuals or physical conditions, and/or in particular that can act more efficiently in stimulating and/or activating relevant neurons, muscles, and/or surrounding areas, and/or enhancing arterial blood flow in various part of the body for muscle recovery of various sizes of muscles and muscle groups.

At least one aim of the invention is to obviate or at least mitigate one or more drawbacks of prior art, and/or address the need for improved reciprocating solutions. A further objective is for the reciprocating solution to be convenient and easy to use, long-lasting, reliable, and/or run smoothly and/or quietly.

### SUMMARY

According to a first aspect of the invention, there is provided a reciprocator device for acting upon a region or part of a human or animal body for producing at least one effect thereupon, which effect can for example comprise any one or more of: stimulating or activating blood circulation; stimulating or activating neurons; stimulating, activating, exercising, or massaging or improving recovery of muscles and/or other tissue; improving performance or function of the body or bodily processes; activating or stimulating male reproductive muscles and/or parts; therapy; and/or medical treatment; the device comprising: a device body; an adjustable drive mechanism; a movable part coupled to the drive mechanism, the drive mechanism being configured to move the movable part reciprocally back and forth with respect to the device body; and the drive mechanism being user-adjustable with the drive mechanism in operational or ready-to-operate condition, to vary the obtainable range of travel of the movable part.

By being user-adjustable with the drive mechanism in operational or ready-to-operate condition, the adjustment to the drive mechanism can take place advantageously when the drive mechanism is ready to operate or is operating, i.e. producing reciprocation. Thus, the range of travel can in various examples be varied instantaneously or at the option of a user whenever desired or selected, for example during reciprocation or without significant pausing of reciprocation, and/or without additional tooling or intervention, and/or without dismantling or reinstating back to the operational or ready-to-operate condition. The device may therefore be well suited for use in therapy situations such as short sessions with patients as it can allow quick or immediate changing of the reciprocation rate and/or effect produced.

The “drive mechanism in operational or ready-to-operate condition” refers to the drive mechanism being complete and functional in the normal manner for producing the reciprocation. In this way, the drive mechanism in operational or ready-to-operate condition can remain intact and provide a functional drive train between the motor and the movable part for moving the movable part in the normal manner.

Preferably, the device further comprises adjuster means to adjust the drive mechanism to vary the range of travel. The drive mechanism may be adjusted such that it obtains a different configuration. For example the drive mechanism can be reconfigurable using the adjuster means between a first configuration in which the movable part is drivable with a first range of travel and a second configuration in which the movable part is drivable with a second range of travel that is different to the first range. The adjuster means can be user operable through suitable control means. The adjuster means can adjust the drive mechanism to vary the range of travel simultaneously to the drive mechanism operating with the movable part moving reciprocally back and forth.

Preferably, the adjustable drive mechanism comprises: a motor; a rotary mover arranged to be driven and rotated by the motor; an adjustable support member; a link member supported upon the adjustable support member via a rotational coupling; a first coupling member arranged to extend between the link member and the rotary mover, the first coupling member having a first end which is coupled to the rotary mover and a second end which is coupled to the link member; and a second coupling member arranged to extend between the link member and the movable part, the second coupling member having a first end which is coupled to the link member and a second end which is coupled to the movable part; wherein the support member is positionally adjustable by the adjuster means to move the rotational coupling between a first location and a second location, for varying the range of travel obtainable by the movable part when operating the rotary mover.

Typically, the first and second locations are end locations, and the adjuster means is operable to move the support member to any suitable location between the end locations, while operating the drive mechanism and whilst reciprocating movement of the movable part is taking place.

Typically, the adjuster means is coupled to the support member through a gear arrangement or a screw arrangement operable to urge the adjustable support member from one location to another relative.

The support member may be supported on the device body. The device body may comprise a casing, e.g. cylindrical casing, and the support member may be supported on a chassis or plate or other structure connected or affixed to the device body. The adjuster means may then be arranged to move the support member with respect to the device body.

Typically, the reciprocator device further comprises control means for operating either or both the adjuster means and the drive mechanism, the control means being operable by a user. The control means may be operable by the user by means of one or more user-operable control devices.

The control means may comprise any one or more of any of: operable buttons, switches, dials; wheels; and/or remote-control electronic devices; manual devices. Any of the control means may be operable by the user. The controls may be operate to determine the rate of reciprocation, e.g. by controlling the motor speed of the motor of the drive mechanism; start or stop reciprocation, e.g. by stopping or starting the motor of the drive mechanism; adjust the range of travel of the reciprocating movable part while reciprocating, e.g. by reconfiguring the drive mechanism from one configuration to another, e.g. by moving the support member from one location to another, thus positioning support member in a different location within the drive part so that a different range of motion is obtained from the drive mechanism in the one configuration as compared with the other. Thus, by way of the control means and/or the adjuster means, the distance of travel of the movable part, or an element connected to the movable part such as a massage head or other head, relative to the device body can be varied according to requirements and/or user preference or wishes. Moreover, the adjustment may be carried out without stopping the motor or dismantling the drive mechanism. Thus, the adjuster means may be operated to move the support member to any suitable position between end positions, during operation of the drive mechanism, more specifically whilst the reciprocating movement of the movable part is taking place, using the control devices, e.g. buttons or remote control.

Typically, the movable part may be a linear drive part arranged to be movable linearly back and forth. The movable part may typically comprise at least one element or head to interact with the human or animal body, in use, the element or head being any one or more of: a stimulator structure; a massage head; a push/pull head; a cup structure. The massage head can have any suitable form.

The element or head may be detachable. The movable part may comprise a stem and the detachable element or head may be detachable from the stem. The element may comprise a head or tool or means configured to be coupled to or located in mechanical communication with said region or part of the body. Thus, the element or head may reciprocate back and forth and may exert or transmit force on said region or part of the body to obtain the desired effect upon the body.

The device is preferably a hand-held device. The device body may comprise a hand-held casing in which the drive mechanism may be housed.

In various embodiments, a variable and/or adjustable travel length of the drive part can be advantageous in that the device may be used to produce a more optimised effect upon the body. The travel length or range of travel may be varied for different muscle sizes or condition of body or configuration of the part of the body to which it is to be applied in use. In the case of a cup/structure which may be sealably located against the body in vacuum lock relationship, the variation in travel length or range of travel of the drive part may optimise the pull push effect produced on the body for enhancing arterial blood flow. With the attachable element being a cup, structure or other element that is located on the body, e.g. in vacuum lock relationship, the drive mechanism can advantageously impart push/pull forces to the body or part thereof in use.

The reciprocator device of the first aspect of the invention may have one or more further features as set out in any of the following second to fifth aspects of the invention.

According to second aspect of the invention, there is provided a massager device comprising: a device body; a drive mechanism; a movable drive part with a massage head coupled thereto; the movable drive part being operatively coupled to the drive mechanism, the drive mechanism being configured to operate to move the drive part and massage head reciprocally back and forth with respect to the device body; and the drive mechanism being user-adjustable with the drive mechanism in operational or ready-to-operate condition to vary the obtainable range of travel of the movable drive part and massage head.

Preferably, the massager device further comprises adjuster means to adjust the drive mechanism to vary the range of travel.

Preferably, the drive mechanism comprises: a motor; a rotary mover arranged to be driven and rotated by the motor; an adjustable support member; a link member supported upon the adjustable support member via a rotational coupling; a first coupling member arranged to extend between the link member and the rotary mover, the first coupling member having a first end which is coupled to the rotary mover and a second end which is coupled to the link member; and a second coupling member arranged to extend between the link member and the movable part, the second coupling member having a first end which is coupled to the link member and a second end which is coupled to the movable drive part; wherein the support member is positionally adjustable by the adjuster means to move the rotational coupling between a first location and a second location, for varying the range of travel obtainable by the movable drive part when operating the rotary mover. The adjustment using the adjuster can in this way take place with the drive mechanism in operational or ready-to-operate condition.

Preferably, the adjuster means is coupled to the drive mechanism through a gear arrangement or screw arrangement operable to urge the adjustable support member from one location to another.

Preferably, the massager device further comprises control means for operating the adjuster and/or the drive mechanism. The control means may typically be operable by a user by means of one or more user-operable control devices. Thus, the distance or range of travel of the drive part and massage head relative to the device body can be varied according to requirements and/or user preference or wishes. Moreover, the adjustment may be carried out with the drive mechanism in operational position. The adjustment may be carried out without stopping the motor or dismantling the drive mechanism. Thus, the adjuster means may be operated to move the support member of the drive mechanism to any suitable position between end positions, during operation of the drive mechanism, more specifically whilst the reciprocating movement of the drive part is taking place, using the control devices, e.g. buttons or remote control.

The massage device is preferably a hand-held device. The device body typically comprises a hand-held casing in which the drive mechanism is housed. The drive mechanism typically comprises an adjuster means, and the massage device is configured to be operable by way of the adjuster means to adjust the distance of travel of the movable drive part and massage head with respect to the device body. The massage device may be termed a massage gun.

In various embodiments, a variable and/or adjustable travel length of the drive part can be advantageous in that the device may be used to produce a more optimised effect upon the body. The travel length may be varied for different muscle sizes or condition of body or configuration of the part of the body to which it is to be applied in use. In the case of a massage head for example, the travel length may be varied for different muscle sizes or condition of body, and or to vary the stimulation effect produced.

The massager device according to the second aspect of the invention may have one or more further features as set out in relation to the first aspect of the invention or any of the following third to fifth aspects of the invention.

According to a third aspect of the invention, there is provided a reciprocator device for acting upon a region or part of a human or animal body for producing at least one effect thereupon, the reciprocator device comprising: a device body; a structure and sealing means, for sealably enclosing an external part or region of skin of the human or animal body; a movable part which is arranged to extend from a section of the device body, the movable part being reciprocally movable to travel back and forth with respect to the device body; the movable part for exerting a force upon the enclosed external part or region of the human or animal body, when the structure in use is sealably located thereupon, in dependence upon the reciprocating movement back and forth; and means to produce the reciprocating movement of the movable part back and forth with respect to the device body.

Typically, the structure for sealably enclosing the external part or region of the human or animal body comprises any of: a ring portion; a bell or cone structure; a cup structure. Said region may be a region of skin, typically.

Typically, the structure, e.g. the ring portion, the bell or cone structure, or the cup structure, includes sealing means to facilitate sealing between the structure and the body part or region to which the structure is applied. The sealing means may comprise an elastomer material. The sealing means may act for preventing or restricting air ingress into a region between the movable part and the enclosed body part or region.

Typically, the structure is configured to be applied to the region, e.g. the region of skin, or part of the human or animal body so that the movable part lockably couples in use to the enclosed part or enclosed region, e.g. the region of skin, through a vacuum lock effect.

Typically, the structure includes an escape passageway through a wall of the structure for letting out fluid or air of the region between the movable part and the sealably enclosed body part or region, e.g. the region of skin. The escape passageway can be configured to be coupled to a pump device for extracting the air from the region between the movable part and the sealably enclosed body part or region, e.g. the region of skin.

Typically, the structure comprises a push/pull head for exerting a push/pull force upon the enclosed part or enclosed region of the human or animal body when sealably located thereupon, in dependence upon the reciprocating movement back and forth.

In certain examples, the movable member comprises an end portion operable for exerting the push/pull force upon the enclosed part or enclosed region, e.g. the enclosed region of skin, in dependence upon the reciprocating movement back and forth.

In certain examples, the structure comprises: an outer portion for enclosing the external part or region, e.g. the region of skin, and the movable part comprising an inner portion arranged insider the outer portion, the inner portion being movable relative to the outer portion, the inner portion being arranged to act upon the enclosed external part or enclosed region; a flexible diaphragm for coupling the outer portion to the inner portion to permit relative movement between the inner and outer portion; wherein the inner portion is an end portion of the movable part, and wherein the outer portion is a ring portion.

In certain examples, the structure, e.g. the ring portion, bell or cup structure, may comprise a lip or edge for kiss contacting the skin or part of the body. The movable part can have an end portion arranged to act in use upon an area of the body, e.g. an area of skin, within the lip or edge contact. The lip or edge can comprise an elastomer material to facilitate sealing against the exterior, e.g. skin exterior. The edge may be sealed by tape or the like. The bell or cup structure may be a rigid bell or cup structure. The reciprocating movement may provide continuously alternating push/pull effect upon the region, e.g. region of skin, or part within the sealed enclosure. The movable part may be movable relative an outer portion of the structure.

In certain examples, e.g. when placed against a generally flat area of the body, e.g. an area of skin, the pull cycle of the movable end may impart suctional force which may pull the area enclosed, and the push cycle of the movable end may in such variants facilitate to push against or impart a push force toward the area. In this way, the structure of this device may be used on the body for providing vacuum therapy.

In certain examples, the structure comprises at least partially flexible walling. In certain examples, the structure can in other variants be substantially rigid.

In certain examples, the structure comprises a rigid cup structure adapted to receive an end of a penis. The structure in such examples can further comprise a sealing sleeve for sealably coupling the cup structure to the end of the penis with the penis inserted, so as to grip the end of the penis with the end of the penis fully inserted at the base end of the cup structure. The bell or cup structure may be secured so as to grip the end of the penis. The reciprocating movement may then produce push/pull movement of the gripped end of the penis. The rigid cup structure can thus be sealably provided on the end of the penis such that the penis head is locked to the rigid cup structure through a vacuum lock effect for exerting a push/pull force upon the penis head upon reciprocation of the rigid cup back and forth.

The produced effect upon the human or animal body can for example comprise any one or more of: stimulating or activating arterial blood circulation; stimulating or activating neurons; stimulating, activating, exercising, or massaging or improving recovery of muscles and/or other tissue; improving performance or function of the body or bodily processes; activating or stimulating male reproductive muscles and/or parts; therapy; anti-aging; cellulite reduction; and/or medical or cosmetic treatment.

Preferably, the means to produce the reciprocating movement back and forth comprises a drive mechanism or other actuator. The drive mechanism is preferably user-adjustable with the drive mechanism in operational or ready-to-operate condition. The drive mechanism can comprise: a motor; a rotary mover arranged to be driven and rotated by the motor; an adjustable support member; and a link member supported upon the adjustable support member via a rotational coupling; a first coupling member arranged to extend between the link member and the rotary mover, the first coupling member having a first end which is coupled to the rotary mover and a second end which is coupled to the link member; and a second coupling member arranged to extend between the link member and the linear mover, the second coupling member having a first end which is coupled to the link member and a second end which is coupled to the movable part; wherein the support member is positionally adjustable to move the rotational coupling between a first location and a second location, for varying the range of travel obtainable by the movable part when operating the rotary mover. In this way, using the adjuster means

Typically, the device includes adjuster means for adjusting the drive to vary the range of travel of the movable drive part back and forth. The adjuster means may move the support member from one position to another for varying the range of travel. The adjuster means may in this way be operated to perform the adjustment with the drive mechanism in operational position. The adjuster means may be operable to adjust the drive mechanism through a screw and thread arrangement or a gear arrangement. For example, the gear arrangement may comprise a gear arranged to engage a tooth track to urge the adjustable support member from one position to another upon rotation of the gear. The gear arrangement may comprise a worm gear which engages with a worm wheel.

The adjuster means may be coupled to the adjustable support member of the drive mechanism. Typically, the adjuster means is operable to positionally adjust the adjustable support member between a first location and a second location for varying the range of travel obtainable by the movable part when operating the rotary mover. The adjuster means may operate to urge the adjustable support member to move the rotational coupling, via which link member may be coupled to the support member, between the first and second locations.

The reciprocator device of the third aspect of the invention may have one or more further features as set out in relation to the first or second aspect of the invention, or the following fourth or fifth aspect of the invention.

According to a fourth aspect of the invention, there is provided a drive mechanism comprising: a motor; a rotary mover arranged to be driven and rotated by the motor; a linear drive part arranged to be movable linearly back and forth; an adjustable support member; and a link member supported upon the support member via a rotational coupling; a first coupling member arranged to extend between the link member and the rotary mover, the first coupling member having a first end which is coupled to the rotary mover and a second end which is coupled to the link member; and a second coupling member arranged to extend between the link member and the linear mover, the second coupling member having a first end which is coupled to the link member and a second end which is coupled to the linear drive part; wherein the support member is positionally adjustable to move the rotational coupling between a first location and a second location, for varying the range of travel obtainable by the linear drive part when operating the rotary mover. More specifically, the drive mechanism may so be adjustable in operational or ready-to-operate condition, e.g. when operating or when ready to be operated for reciprocation in a reciprocating device or massager device, e.g. such as described anywhere herein.

Typically, the drive mechanism comprises a gear arrangement operable by means of adjuster means to urge the adjustable support member from one position to another. The gear arrangement may comprise a gear arranged to engage a tooth track to urge the adjustable support member from one position to another upon rotation of the gear. Alternatively, the drive mechanism may comprise a screw and thread arrangement operable by means of adjuster means to urge the adjustable support member from one position to another.

The drive mechanism may further comprise a motorised adjuster means for urging the support member from one position to another. In embodiments where a gear arrangement is used, the adjuster means may be configured to operate the gear arrangement to move the support member. The drive mechanism may further comprise operating means for operating the adjuster means, being any one of: lever, switch, mechanical or electrical button, remote transmitter. In embodiments where a screw and thread arrangement is used, the adjuster means may be configured to turn the screw in engagement with the thread to move the support member. The screw may be turned manually or by electrical motor. In manual operation, the adjuster means may comprise a wheel which is hand movable by the user. The wheel can thus be accessible through a product casing so as to be operable without taking the casing apart. The adjuster means may be operable to turn the screw and adjust the position of the support member when the device is in operational or ready-to-operate condition, e.g. when ready to operate or when operative, i.e. when the device is moving the drive part reciprocally back and forth.

Typically, the support member is configured to be adjustable to move the rotational coupling from one location to another when the rotary drive motor is operating or when the rotary drive motor is ready to operate and produce reciprocation.

Typically, the rotational coupling comprises a hub received in an aperture in bearing relationship so that the link member is supported in bearing relationship, the hub rotatable relative to the aperture on the support member.

Typically, the first end of the first coupling member is coupled to the rotary mover via a first coupling; the second end of the first coupling member is coupled to the link member via a second, rotational coupling; the link member is coupled to the support member via a third, rotational coupling; the first end of the second coupling member is coupled to the link member via a fourth, rotational coupling; and the second end of the second coupling member is coupled to the drive part via a fifth, rotational coupling.

In the first location, typically the first and second coupling members are operable to move the linear drive part linearly with a first range of travel upon rotation of the rotary mover; and in the second location, typically the first and second coupling members are operable to move the linear mover linearly with a second range of travel upon rotation of the rotary mover.

Typically, the drive mechanism comprises means for mounting the drive mechanism to a device body, e.g. a body of a product. The means may comprise a supporting structure, frame, base or chassis. The support member may be movably mounted to a supporting structure which may be adapted to be mounted to the device body, e.g. the body of a product. The supporting structure may in some examples comprise or be a base plate.

Typically, the drive part is mounted in channel and is movable linearly to extend from and retract into the channel in dependence upon the extent of travel.

The drive mechanism of the fourth aspect of the invention may have one or more further features as set out in relation to any of the first to third aspects of the invention.

According to a fifth aspect of the invention, there is provided a reciprocator device for acting upon a region or part of a human or animal body for producing at least one effect thereupon, the device comprising: a device body; a drive mechanism in accordance with the fourth aspect of the invention; and a movable part coupled to the drive mechanism, the drive mechanism being configured to move the movable part reciprocally back and forth with respect to the device body. The reciprocator device of the fifth aspect of the invention may have one or more further features as set out in relation to any of the first to fourth aspects of the invention.

Any of the aspects of the invention may include any one or more further features as described anywhere else herein or as described in relation to any other aspect wherever described herein.

Embodiments of the invention can be advantageous in various ways as will be apparent from the specification throughout.

### BRIEF DESCRIPTION OF THE DRAWINGS

There will now be described, by way of example only, embodiments of the invention, with reference to the accompanying drawings, in which:

FIG. 1 is a side-sectional representation of a reciprocator device in the form of a massager comprising a massage head arranged to be reciprocated linearly back and forth and applied to an external region of a person's body for massaging the body;

FIG. 2 is a side view representation of the device of FIG. 1;

FIGS. 3A to 3C are side representations of different heads for the device of FIGS. 1 and 2;

FIG. 4 is a side-sectional representation of another device, for application to a person's body for producing an effect on the body, the device having a head in the form of a sealable cup or bell structure;

FIG. 5 is a side view representation of the device of FIG. 4;

FIG. 6 is a close-up perspective representation of a variant of the head for the device of FIGS. 4 and 5, additionally including an arrangement for urging a sealing ring portion against the skin of the body;

FIG. 7 is a perspective view of a configurable and adjustable drive mechanism in accordance with an embodiment of the invention for producing reciprocating motion of a drive part;

FIG. 8 is a side view of the drive mechanism of FIG. 7;

FIGS. 9A & 9B are side view representations of the drive mechanism of FIGS. 7 and 8 at respective end positions of the linear travel of the drive part in a first, long travel configuration of the drive mechanism;

FIGS. 10A & 10B are side view representations of the drive mechanism of FIGS. 7 and 8 at respective end positions of the linear travel of the drive part in a second, short travel configuration of the drive mechanism;

FIG. 11 is a perspective view of the drive mechanism from a different angle;

FIG. 12 is a perspective representation of the drive mechanism of FIG. 8 from a different angle;

FIG. 13 is a top view representation of the drive mechanism of FIGS. 8 and 9 with an arrangement of batteries on top;

FIG. 14 is a perspective representation of alternative adjuster means for adjusting the position of the support member for reconfiguring the drive mechanism;

FIGS. 15A-15C are schematic representations of another push/pull stimulator head illustrating the principle of operation in different phases of movement; and

FIG. 16 is a schematic representation of another reciprocator device with a push/pull head comprising a rigid cup structure.

### DETAILED DESCRIPTION OF THE DRAWINGS

### Reciprcator Devices

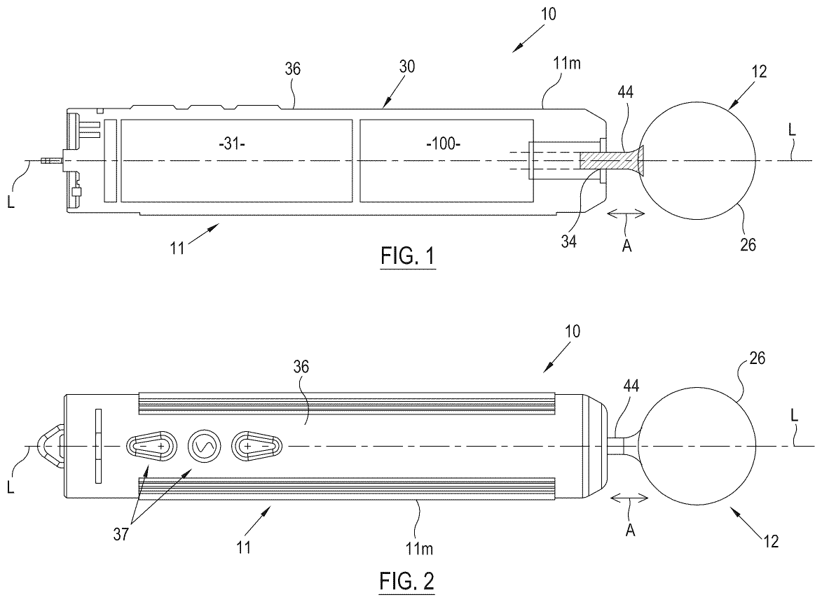

With reference first to FIGS. 1 and 2, a reciprocating, massage device 10 for performing massage is depicted, in use. The massage device 10 has generally two parts, a first part 11 and a second part 12. The second part 12 is a part arranged to be reciprocated by the first part 11 and to be applied to the exterior of a human or animal's body to produce an effect thereupon. The first part 11 comprises an operating unit 30 that includes the means to produce reciprocating movement of the second part 12, relative to the first part 11, as indicated by arrows A.

The second part 12 in this example comprises a massage head 26, which in the example is spherical.

Thus, when the operating unit 30 of the device 10 is held in the operator's hand and the device 10 is operated, reciprocating movement of the massage head 26 is produced relative to the first part 11, and the massage head 26 contacting a part or region of a person's body can exert a force upon the person's body in dependence upon the reciprocation.

The first part 11 of the device 10 comprises in this example an operating unit 30 for operating the device 10 and producing the reciprocating movement of the massage head 26 in use. The operating unit 30 has a device body and includes a drive mechanism 100 through which the rotary motion of an electric motor is transmitted and transformed into a linear reciprocating motion of a movable drive part relative to the device body. The drive mechanism 100 is housed within the device body of the operating unit 30, the device body being in the form of cylindrical housing 36. The operating unit 30 includes various electronics and control means 31 for operating the drive mechanism 100, which are also housed in the cylindrical housing 36. Hand operable controls 37 are provided on the housing 36 for the user to adjust the rate (stroke rate) and extent of travel (stroke length) of the reciprocation of the drive part.

The massage head 26 is provided on a stem 44, by which the massage head 26 is coupled to the operating unit 30, and more specifically the drive mechanism 100. The stem 44 is an extension of the drive part of the drive mechanism 100, or can in other variants constitute the drive part, and is movable linearly back and forth by operation of the drive mechanism 100. The stem 44 (which is movable by the drive mechanism) extends from an end of the cylindrical housing 36. The stem 44 is further located in a channel 34 in an end portion of the housing 36 and is arranged to be movable along the channel 34 relative to the housing 36. The operating unit 30 includes the drive mechanism 100 and the drive motor (described further below) for operating the drive mechanism 100. The drive mechanism 100 is operable by this motor to move the stem 44 (and consequently the massage head 26) reciprocally and linearly back and forth with respect to the housing 36 of the first part 11, as indicated by the arrows A.

The operating unit 30 in this example is configured to be hand-held and hand operable. More specifically, a user can grip around the housing 36 of the operating unit with one hand and operate manual controls 37 for operating the device, using digits of the same hand. For example, the housing 36 has switches and/or buttons 37 for activating the drive mechanism and/or varying the rate of reciprocation and/or the varying the distance of travel of the stem 44 and hence the massage head 26 between end points in its movement cycle. These buttons and/or switches 37 are hand operable. For example, at one setting, the stem 44 may travel 15 mm in each direction back and forth, and at another setting, the stem 44 may travel 5 mm in each direction back and forth. The speed of reciprocation is also adjustable. At one setting, the cycle of travel back and forth is shorter or longer than at another setting, adjustable according to requirements and/or user preferences or choice. In other variants, app-based remote-control means are used instead of the manually operated buttons and/or switches 37 on the operating unit 30.

Electronics unit 31 is also located in the housing 36 of the operating unit 30. The electronics unit 31 in this example has one or more electrical devices to perform an adjustment of the drive mechanism. The one or more electrical devices can include a servo for turning a gear of a gear arrangement (as will be described further in the following). The electronics unit 31 also includes batteries for providing power, e.g. to operate the servo and/or the drive motor of the drive mechanism 100. The electronics unit 31 also includes circuitry for communicating or receiving a signal, e.g. from the switches and buttons 37, for operating the one or more electrical devices to perform an adjustment or for stopping or starting the reciprocation.

Alternatively, as will also be exemplified further below, the adjustment may be carried out manually, for example the operating unit 30 may be provided with a wheel operable by the user's hand when holding the operating unit in the same hand, such that turning the wheel turns a screw which adjusts the drive mechanism from one configuration to another. The adjustment can take place while the drive mechanism operational, e.g. when it is ready to operate or when operative, i.e. whilst the device is reciprocating.

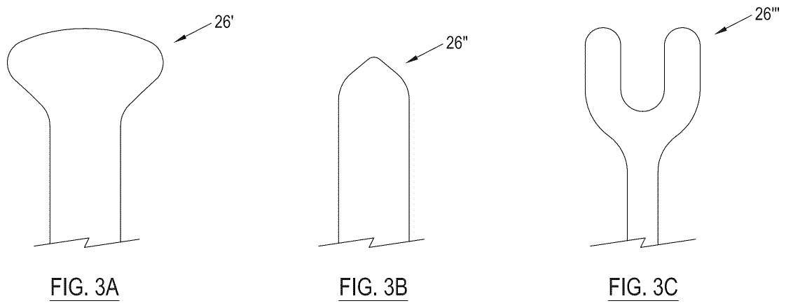

Turning to FIGS. 3A to 3C, different massage heads—26′, 26″, and 26″ can be seen and used in other variants instead of the spherical massage head 26 depicted in FIGS. 1 and 2. In FIG. 3A, a mushroom massage head 26′ is depicted, in FIG. 3B a dart massage head 26″ is depicted, and in FIG. 3C a fork massage head 26″′ is depicted.

By using the massage device 10 of FIGS. 1 and 2, the amount of travel of the movable part 44, i.e. the stroke length, can be varied and adjusted according to needs. This along with a suitable choice of massage head (see those for example in FIGS. 3A to 3C) may allow the massage to be configured as required. The rate of reciprocation, i.e. the stroke rate, may also be varied conveniently to further customise the massage provided by the device. The stroke rate and stroke length can be varied smoothly without stopping the action of the massager device 10. The drive mechanism 100 is configured and adjustable as described in further detail below. In this example, the means of providing the reciprocation comprises the drive mechanism 100 but in other variants this can comprise electro-magnetic devices, or other actuator devices.

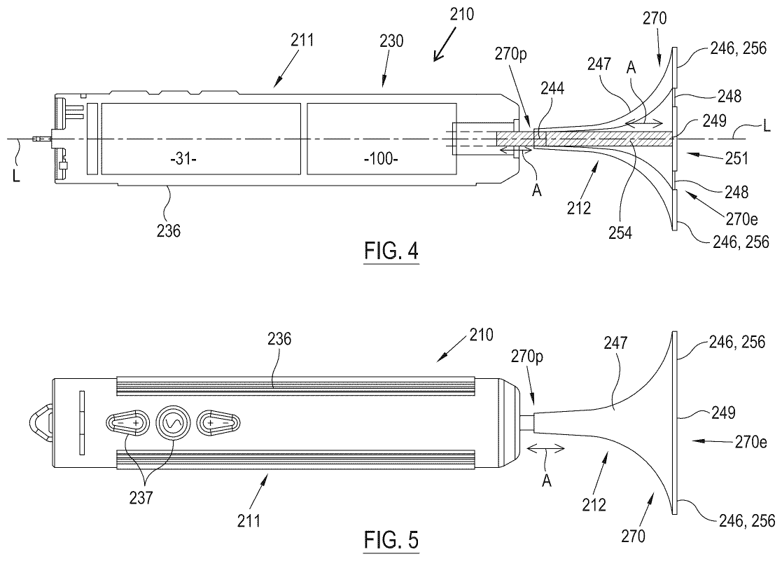

With reference now to FIGS. 4 and 5, another reciprocating device 210 is depicted. Certain features which are similar to those of the device 10 have the same reference numerals incremented by two hundred. Generally, the reciprocating device 210, has two parts 211, 212 where the one part is arranged to be movable back and forth reciprocally relative to the other by operation of the drive mechanism 100 of the operating unit 230. In this example, instead of operating as a massage device, the second part 212 comprises push-pull stimulator head 270.

The push/pull stimulator head 270 comprises an outer body 247 which extends in an axial direction from a location proximate to the first part 211 of the device 210 toward a head end 270e, distal therefrom. The outer body 247 has an outer wall section that curves outwardly and increases in diameter toward the head end 270e. At the head end, the outer body 247 defines an outer ring 246 which is to be arranged in contact against the skin of a user. The outer body 247 is provided with sealing means 256 for forming seal between the outer ring 246 and the skin.

The push/pull stimulator head 270 further comprises an inner body 251 arranged inside the outer body 247. The inner body 251 has a shaft 254 which extends distally toward the head end 270e and a push/pull member 249 arranged on the shaft 254 at the head end. The push/pull member 249 in this example is disc-shaped plate arranged concentrically inside the outer ring 246. The plate extends radially from the shaft 254. The push/pull member 249 is also arranged to contact the skin of the user in use.

The inner body 251 is connected to the outer body 247 via diaphragm 248 extending radially between the inner, push/pull member 249 and the outer ring 246.

The shaft 254 of the inner body 251 is connected to the stem 244 so as to form an extension of the stem 244 which is movable back and forth from the housing of the first part 211 of the device. The shaft 254 is disconnectable from the stem 244 for removal of the push/pull head 270 if or when required or desired.

The outer body 247 fits around the shaft 254 and/or the stem 244 at the proximal end 270p, allowing movement of the stem 244 and/or shaft 254 back and forth relative to the outer body 247 at the proximal end 270p.

In use, upon reciprocation of the movable stem 244 and shaft 254 as indicated by arrow A, the push/pull member 249 is moved back and forth to exert force against the area of skin enclosed by the outer body 247 of the push/pull stimulator head 270. The area of skin is sealably enclosed by the outer ring 246 of the outer body 247 and the sealing means 256 forming a seal against the skin. The end plate of the push/pull member 249 contacts the area of the skin, thus stimulating the skin and/or underlying tissue by action of reciprocating the end portion 249 of the movable stem 244 back and forth. When the push/pull stimulator head is applied to the body and the surfaces of the ring 246 and plate 249 at the distal end 270e are located against the skin and the outer ring 246 sealed by sealing means 256 (e.g. by applied pressure and/or elastomeric seal), a vacuum lock effect is obtained between the push/pull member 249 and the enclosed skin. The diaphragm 248 provides some flexure in the coupling between the push/pull member 249 and the outer body 247 so that the push/pull member 249 can move in and out whilst locked to the skin. Upon travel away from the body, the push/pull member 249 acts to pull on the skin and may “lift” the skin somewhat. Upon travel toward the body, the push/pull member 249 pushes against the skin. The outer ring 246 comprising sealing means in the form of seal ring 256 of elastomeric material which when laid against the skin of the body encircles an area of the skin and seals against the skin. When pushing the push/pull member toward the skin, excess air if inadvertently entered can be forced out of the enclosed region between the distal end 270e and the skin (e.g. through a valvel not shown), to facilitate maintaining a vacuum effect in the region between the distal end 270e and the skin, to facilitate the locking of the inner body 251 to the skin and an effective suctional pull force on the skin when travelling away from the skin. In this way, the inner purh/pull member 249 can produce a pull effect upon the skin alternating with a push effect.

By providing both a push and a pull effect upon the area of skin, the device 210 of FIGS. 4 and 5 may stimulate or enhance blood circulation. The increase in blood circulation when applied to muscles which have been subjected to exercise may facilitate muscle recovery. Using the operating unit 230, the length or range of travel of the movable part 244 may be varied to provide an adjustable effect according to user needs. The reciprocating device 210 uses the same drive mechanism 100 as described in relation to massager device 10 and is configured and adjustable as described in further detail below.

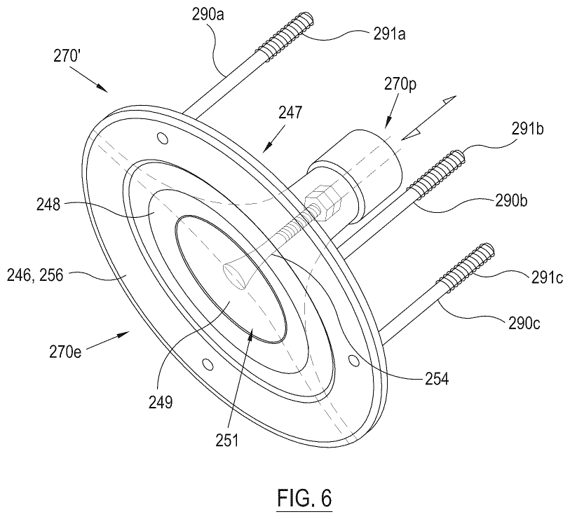

In FIG. 6, a variant push/pull stimulator head 270′ is depicted generally. The variant head 270′ is configured as described above in relation the stimulator head 270 except it also has means for urging the outer body 247 against the skin of the user's body, the means in this example comprising positioner pins 290a, 290b, 290c provided with springs 291a-291c. Each positioner pin 290a, 290b, 290c has first and second ends and at the first end each is coupled to the outer body 254 of the stimulator head 270′ and at the second end is coupled to the housing 230 of the first part 211 of the device 210, via the spring 291a-291c. The spring 291a-291c allows movement between the first and second ends during reciprocation and are biased to extend axially to urge the outer ring 246 of the outer body 254 against the skin during use of the device 210.

In use typically, the outer ring portion 246 is located on the skin body and is stationary. The springs 291a-291c are anchored to the housing body 230 which is also stationary. The springs 291a-291c act between the housing 230 and the outer ring 246, and apply pressure to the outer ring 246, so that it is kept in place in stationary position. The springs can move, e.g. compress or extend, if the angle of the operating unit 230 relative to the body is changed. When anchored to the housing body 230, the springs 291a-291c in this way may apply an even pressure to the ring portion. However, the springs 291a-291c in alternative variants is anchored instead to the outer ring 246.

Advantageously, the urging means can be employed to maintain the position and the sealing relationship of the outer ring 246 against the skin while the inner push/pull member 249 moved back and forth with different ranges of motion. This can facilitate ease of use, e.g. the user may simply need to place the stimulator head 270′ against the user's skin and hold the device with one hand, i.e. hand holding around the casing 236 of the first part of the device, in order to operate. The positioner device with the spring operates to keep the device in place on the skin, and may facilitate sealing of the outer ring against the skin.

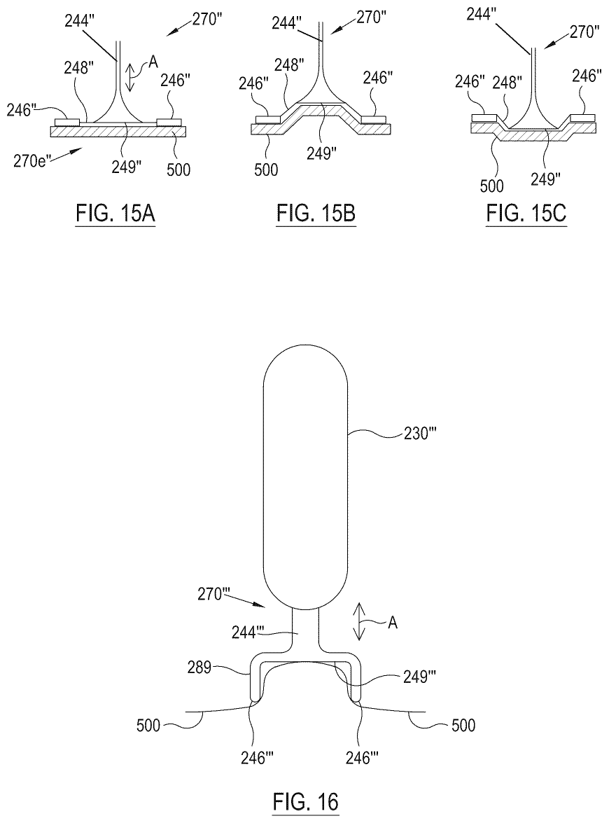

FIGS. 15A to 15C illustrate by way of schematically depicted variant push/pull stimulator head 270″ the manner of operation against the skin 500 of a human being or animal. This operating principle is the same for push/pull stimulator heads 270, 270′ described above. In FIG. 15A, the end 270e″ of the stimulator head 270″ is located flat against the skin 500, in neutral position. The region within the outer ring 246″ is free of air. The outer ring 246″ seals against the skin so as to prevent air entering the region within the outer ring 246″. The inner end surface 249″ abuts and contacts the skin 500 in the region inside the outer ring 246″. The stem 244″ is then activated to move reciprocally back and forth as indicated by the arrow A. In FIG. 15B, as the stem 244′ retracts, the skin 500 in the region within the ring 246 is lifted in accordance with the retracting movement of the stem. The stem 244″ and inner end surface 249″ locks to the skin within the ring 246″ through a vacuum lock effect, with the outer ring 246″ sealing against air outside the head. Thus, the stem 244″ when reciprocating and travelling toward the operating unit (away from the skin) exerts a pulling force on the skin. Conversely, as seen in FIG. 15C, the stem 244″ and end surface 249″ pushes against the skin 500 within the outer ring 246″, when reciprocating and travelling away from the operating unit (toward the skin). The operation of the device with the push/pull stimulator heads 270, 270′, 270″ thus provides push/pull effect upon the skin of the person or animal to which this is applied.

With reference to FIG. 16, a yet further variant of a push/pull stimulator head 270″′ is depicted, coupled to the operating unit 230″′, for reciprocation. In this variant, the push/pull head 270″′ comprises a rigid cup 289 that is disposed on an end of the stem 244″′. The open end of the rigid cup 289 comprises a ring 246″′ and encircles a region of skin 500. The ring 246″′ is sealably coupled to the skin, e.g. through suitable sealing means or seal members, for preventing or restricting air ingress into the cup. A body part may fit into the cup 289 so that the body part or skin thereof in the cup may locate against an end surface 249″′ of the base of the cup. Thus, the cup can connect and locked to the skin 500 or part in the cup with vacuum lock effect being produced therebetween. Upon reciprocating the push/pull stimulator head the rigid cup is moved back and forth as indicated by arrow A, and exerting push/pull forces alternatingly on the body part or skin thereof. In locked relationship of the cup to the body part, a push force is thus produced as the stem 244″′ moves away from the operating unit 230″′ (toward the user's body/skin 500), and the pull force is produced as the stem 244″′ moves toward the operating unit 230″′ (away from the user's body/skin 500).

### Drive Mechanism

The operating unit 30, 230 of the reciprocating devices 10, 210, 210″, 210″′ includes the drive mechanism 100 which will now be described in further detail as an example means for producing reciprocating movement.

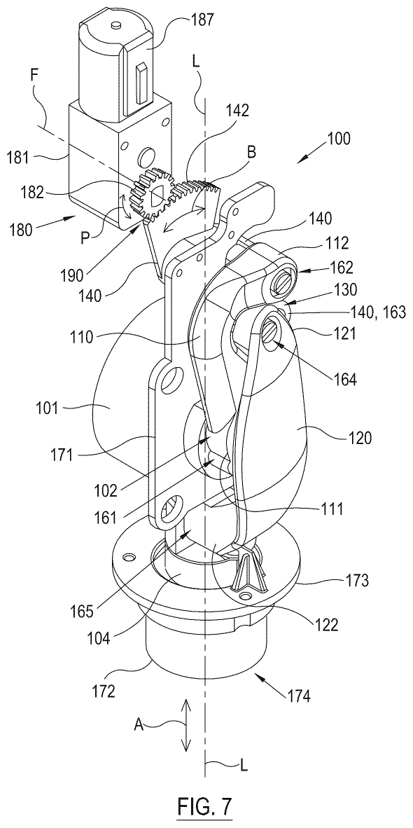

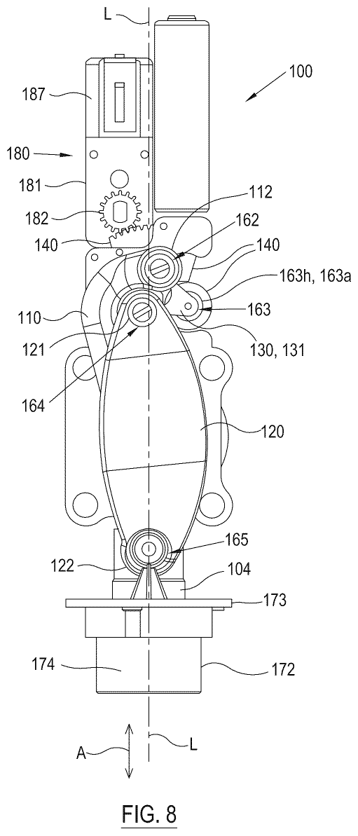

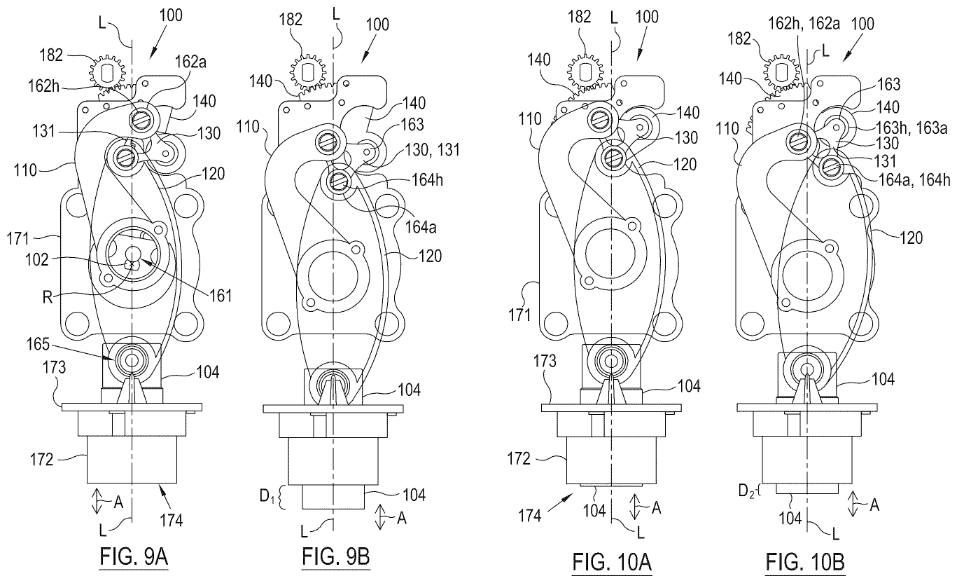

Turning now to FIGS. 7 and 8, a configurable drive mechanism is generally depicted at reference numeral 100. The drive mechanism 100 is configured to produce linear reciprocation of a drive part 104. The drive part 104 is arranged to be movable back and forth in the direction indicated by the arrow A. The drive mechanism 100 has an electric motor 101 which is operable to drive and rotate a rotary mover in the form of rotary shaft 102 (see also FIG. 9A). Further, the drive mechanism 100 includes a first coupling member 110, a second coupling member 120, a link member 130, and an adjustable support member 140.

The first coupling member 110 extends between the rotary driver 102 and the link member 130. The first coupling member 110 has a first end 111 which is coupled to the rotary driver 102, at first coupling 161, and a second end 112 which is coupled to the link member 130, at a second, rotational coupling 162.

The link member 130 is coupled to the adjustable support member 140 at a third, rotational coupling 163.

The second coupling member 120 extends between the link member 130 and the linear drive part 104. The second coupling 120 member has a first end 121 which is coupled to the link member 130, at a fourth, rotational coupling 164, and a second end 122 which is coupled to the linear drive part 104, at a fifth, rotational coupling 165.

The second to fifth rotational couplings 161-165 are rotatable about respective axes perpendicular to the longitudinal axis L and the direction A of linear reciprocation of the linear drive part 104. The axes of rotation of the second to fifth rotational couplings 161-165 are parallel axes.

The adjustable support member 140 is positionally adjustable and movable in an arc as indicated by arrow B. The movable support member 140 is mounted on a base plate 171 which in turn can be fixed to a body of a product. The movable support member 140 is movably mounted to the base plate 171, and thus is movable relative to the base plate 171 as indicated by arrow B. Also conveniently in this example, the motor 101 is mounted to the base plate 171.

As the adjustable support member 140 is moved along the arc, so too the third coupling 163 (between the support member 140 and the link member 130) is moved. In this way, the range of travel obtainable by the linear drive part 104 in response to operation of the rotary driver 102 is variable. The third rotational coupling 163 in its entirety is moved along with, i.e. travels with, the support member 140 between a first location and a second location relative to the base plate 171. The base plate 171 constitutes a chassis that can be fitted and mounted fixedly to the casing 34 of the operating unit. The chassis could take other forms in other variants and may not necessarily comprise a base plate. The third rotational coupling 163 upon movement into a different location supports the first and second coupling members 110, 120 at different distances away from the rotary driver 102. The range of travel obtainable by the linear drive part 104 in response to operation of the rotary driver 102 varies depending upon the position of the support member 140 and the movement of the support member 140 from one position to another, whereby the rotational coupling 163 moved to another location.

The drive part 104 is located in a channel 174 and is arranged to move slidably back and forth along the channel 174. The channel extends longitudinally along axis L. The channel 174 provides support for the drive part 104 laterally and helps to guide the moving drive part 104. The channel 174 in this example extends through a structure 172 comprising a collar 173 which can facilitate its fixing to a body of a product (not shown), and/or to an operating unit of the product such as the operating unit 30, 230.

With reference now additionally to FIGS. 9A and 9B and FIGS. 10A and 10B, the travel of the drive part 104 in different configurations of the drive mechanism 100 can be further appreciated. In FIGS. 9A and 9B, the adjustable support member 140 is in a first position, and in FIGS. 10A and 10B the adjustable support member 140 is in a second position. In the second position, the third coupling 163, that is the coupling of the link member 130 to the support member 140, is moved away from the first location to a second location further along the axis L, so that the range of motion transferable through the link member 130 from the first coupling member 110 to the second coupling member 120 along the axis L is reduced. The drive part 104 is movable reciprocally and linearly back and forth between first and second end positions of each cycle. FIGS. 9A and 10A show the configuration of the drive mechanism at the first end position within a cycle, and FIGS. 9B and 10B show the configuration of the drive mechanism 100 at the second end position within the cycle. When the adjustable support member 140 is in the second position as seen in FIG. 10B, the link member 130 between the first and second coupling members 110, 120 obtains a greater component extent in the direction transverse to the axis L than when the support member 140 is in first position as seen in FIG. 9B.

In one cycle back and forth, the drive part 104 is moved between a first end position and a second end position, and the distance of travel from the first to the second end positions per cycle with the support member 140 in the first position (FIG. 9A) is greater than when the support member 140 is in the second position (FIG. 10A). The extent of the travel and the amount that the drive part 104 extends from the channel 174 can be varied by moving the support member 140 between the first and second positions. As can be seen in FIG. 9B, the drive part 104 travels and extends from the end of the channel 174 by the distance D1, whereas in FIG. 10B the drive part 104 travels and extends from the end of the channel 174 by the distance D₂. The distance D₂ is significantly less than D1.

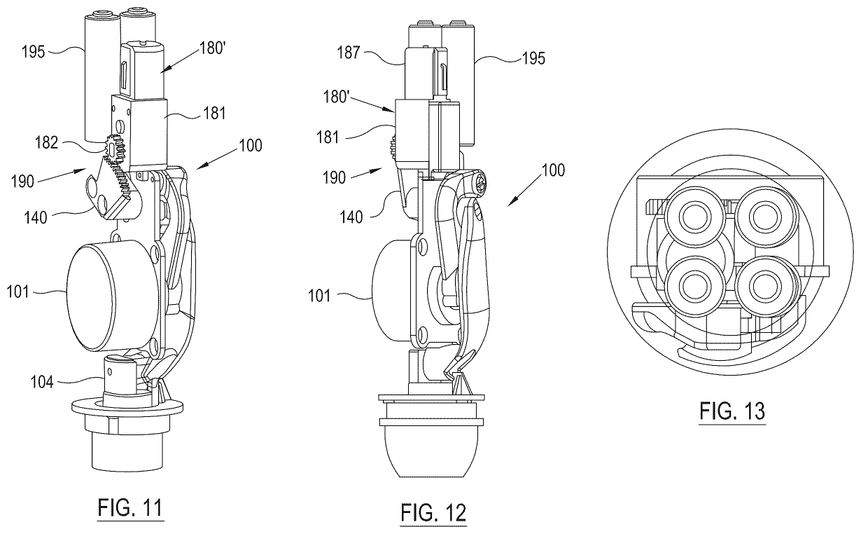

Also seen with further reference to FIGS. 11 to 13, the support member 140 can be adjustably moved into different positions, and in this example the drive mechanism 100 includes an adjuster means 180 for adjustably moving the adjustable support member 140 to different positions. The adjuster means 180 is arranged to engage the support member 140 through a gear arrangement 190. More specifically in the example depicted, the adjuster means 180 comprises an actuator 181, which in this example is a servo motor 187, that drives a pinion gear 182 which in turn engages an arcuate tooth track 142 on the support member 140. The actuator 181 is operable to rotate the pinion gear about a fixed axis F. The actuator 181 is configured to be mounted in fixed position to a body of a product (not shown) or to a component such as a chassis or base plate for fitment to the body of the product for example to the casing body 30, 230 thereof. The pinion gear 182 engages the tooth track 142. Upon rotating the pinion 182 on the tooth track 142, the support member 140 is urged from one position to another, relocating the coupling 163 of the link member 130 to the support member 140.

In other variants, the support member 140 is moved between positions in other ways, for example using other means than gears for engaging and/or moving the support member 140. The gear arrangement 190 can also take other forms.

The servo motor 187 is an electrical motor. The electrical servo motor 187 is controllable and can be activated for movement of the pinion 182 in one direction or the other depending upon the adjustment to be made. The electrical servo motor 187 in some variants is controllable by user controls, e.g. by buttons, switches, or the like. The electrical servo motor 187 in some variants is operable in accordance with pre-programmed instructions. The motor in some variants is operable by remote control. Activating the servo motor 187 operates the actuator 181 to adjust the support member 140 of the drive mechanism 100 to vary the extent of linear travel of the drive part 104.

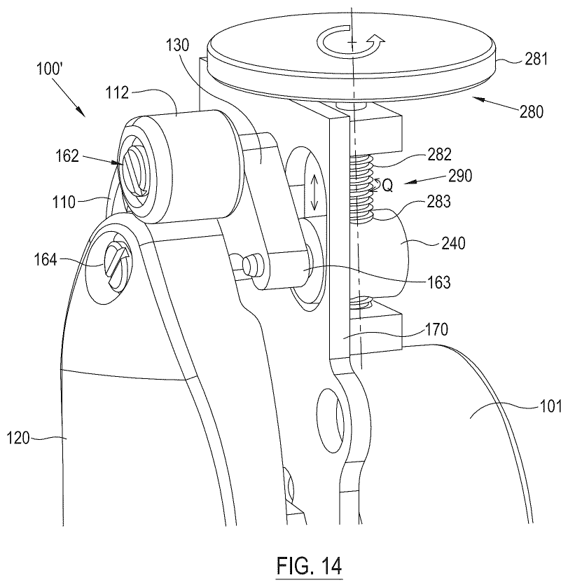

In FIG. 14, an example variant drive mechanism 100′ is depicted having an alternative adjuster means 280 is provided. The link member 130 in this variant is connected to the adjustable support member 140 through rotational coupling 163. In this case, the adjustable support member 140 has a cylindrical body located in an elongate slot in the plate 170. With the cylindrical body arranged in the slot, the support member 240 is movable to travel up/down along the slot, and in turn thereby moves the position of the rotational coupling 163 to different location where the drive part 104 has a different range of travel. Instead of the gear arrangement, the adjuster means 280 includes a screw 282 arranged to engage the thread of a threaded hole 283 in the cylindrical body of the support member. By turning the screw 282 in the hole 283, the cylindrical body and support member travels axially along the screw and along the slot to change the position of the rotational coupling 163. The adjuster means 280 further has a control dial connected to the screw, so that turning the dial turns the screw up or down, operable manually by a user of the device. For example, the dial may extend through the casing, so that the dial may be accessed and hand operated on the exterior of the operating unit 30, e.g. instead of one or more of the buttons 37, while holding the operating unit 30 when the device is in use.

The drive motor 101 for producing the reciprocation of the drive part 104 of the drive mechanism 100 is electric and is also controllable to operate at different speeds. The drive motor 101 is controllable by user controls, e.g. by buttons, switches or the like, provided on the operating unit. The electrical drive motor 101 in some variants is operable in accordance with pre-programmed instructions. The motor 101 in some variants is operable by remote control. A higher speed of the motor 101 increases the rate of linear reciprocation of the drive part 104.

The first coupling, between the first end 111 of the first coupling member 110 and the rotary mover 102 is fixed coupling, where the first end 111 is arranged eccentrically with respect to the axis of rotation R (see FIG. 9A) of the rotary mover 102. The first end 111 orbits therefore around axis R and the coupling member 110 has a component of movement along the long axis L. Furthermore, the arm extends between the first end 111 and the second end 112 on curved “swan-neck” trajectory. This facilitates coupling to the link member 130 beyond the first end 121 of the second coupling member 120. The second end 112 upon rotation of the rotary movement with a transverse component of movement which is accommodated by the rotational coupling 163 of the link member 130 to the support and the rotational coupling 162 of the second end 112 of the first coupling member 120 to the link member 130.

The link member 130 in the example comprises a triangular plate 131 with the second, third and fourth rotational couplings 162, 163, 164 define corners of a triangle aligned with the three corners of the triangle of the plate 131. The link member 130 has coupling hubs 162h, 164h on the plate which are arranged in a complementary coupling apertures 162a, 164a in the first and second coupling members 110, 120 respectively. The link member 130 also has coupling hub 163h on the reverse side of the plate which is arranged in a complementary coupling aperture 163a in the support member. The coupling hubs 162a, 163a, 164a are supported in the complementary coupling apertures 162a, 163a, 164a on bearings, e.g. roller or ball bearings. These rotational couplings 162, 163, 164 can therefore provide low friction couplings which allow rotation between parts relatively freely and facilitate which can reduce wear. In other variants, the converse coupling arrangement is possible with the coupling hubs 162h, 164h, 163h provided on the coupling members 110, 120 and the support member 140, and the coupling apertures 162a, 163a, 164a provided in the link member 130.

The fifth coupling 165 of rotary type also has a coupling hub on either the drive part 104 or the second end 122 of the second coupling member 120 and a complementary coupling aperture on the other of the drive part 104 and the second end 122, supported in bearing relationship with bearings therebetween, e.g. roller or ball bearings. The couplings 164 and 165 accommodate together with the rotational coupling 163 components of movement imparted from the first coupling member upon rotation of the rotary mover 102 in dependence upon the location of the support member 140. The ball bearing couplings may facilitate durability and smooth and quiet running of the reciprocator device.

In use, a user may activate the device 10 and switch on the motor 101. The motor 101 then rotates the rotary mover 102 at a certain speed. The first coupling member 110 follows the rotation and at the coupling 162 to the link member 130 moves relative to the support member 140, with the movement accommodated by rotation of the link member 130 at the coupling 163 relative to the support member 140, rotation at the couplings 162, 164 between the link member 130 and the first and second coupling members 110, 120, and rotation at the coupling 165 between the second coupling member 120 and the drive part 104 which is constrained in the channel 174 to move in the in the defined linear direction A. A certain extent and distance of travel of the drive part 104 is obtained per cycle back and forth linearly as the rotary mover 102 rotates. The drive part 104 can then be applied to impart reciprocating movement to other components or structures depending upon the product. The motor speed is optionally varied to change the rate of reciprocation. The adjuster means 180 is optionally operated and controlled to move the position of the support member 140 and hence move the location of the coupling 163 between the link member 130 and the support member 140, and thereby vary the rate of reciprocation, which is possible when the motor is running without requiring to first stop the motor 101, i.e. it can be done while the drive part 104 is moving reciprocally back and forth. The drive mechanism is adjustable in operational or ready-to-operate condition, i.e. when it is ready to operate or is operating. The amount of extension of the drive part 104 can therefore be increased or decreased as required.

The drive mechanism 100 can be incorporated into the body of a reciprocator product for example into the hand-held casing 36 of the operating unit, a handle, or another part of the reciprocator product. The coupling members 110 and 120 are arranged side by side in overlap and both extend generally between their first and second ends along the longitudinal axis L.

In FIGS. 11, 12 and 13, the arrangement of the drive mechanism 100 with batteries 195 for operating the motors 101, 187 illustrated. The compact nature can be seen. The drive mechanism 100, adjuster means 180′, and batteries are fitted within a limited diameter as indicated in FIG. 13, yet a robust and reliable reciprocating mechanism can be provided that is configurable to vary the obtainable extent of travel for the linear drive part 140.

Variable stroke rate may be obtained. Stroke rates in the region of 200 to 3000 strokes per minute may be obtained. The speed of the motor 101 determines the stroke rate. The amount of extension obtainable per stroke may typically be in the range of 2 to 18 mm. The drive mechanism 100 is adjustable to any value within that range, using the adjuster means. The adjustment can take place while the motor and drive mechanism is operating i.e. when reciprocation is taking place. Typically, when operating the device for percussive therapy and/or for muscle recovery the range of extension per stroke is 8 to 18 mm. When performing vibrational therapy, the range of extension per stroke is lower such as 2 to 7 mm. The reciprocator devices described herein may be configured to provide a certain range of possible extension, such as those above, or any range within any of those ranges, or any other suitable range, and the reciprocator device in such examples may then be adjustable to any value within the range which the reciprocator device is capable of.

The solution of the drive mechanism described above can advantageously be applied for various reciprocating devices and can be particularly suitable when working to constraints of limited space or limits of acceptable weight or ease of handling. This may be challenging especially so for hand-held reciprocating devices, for example products to be operated by users themselves. Also, durability and robustness of the reciprocating device may also need to meet relevant standards, which can be stringent, depending on the product and application. The design proposed, e.g. through the use of bearings in the various coupling of the drive mechanism and manner of adjustment by adjusting the position of the support member 140, can facilitate smooth operation to avoid vibrations and noise, and improve wearability and longevity.

claims

1

.-

25

. (canceled)

26. A reciprocator device for acting upon a region or part of a human or animal body for producing at least one effect thereupon, the device comprising:
a device body;
an adjustable drive mechanism, the drive mechanism comprising:
a rotary motor for operating the drive mechanism; and
a rotary shaft arranged to be driven and rotated by the motor;
a movable part coupled to the drive mechanism;
the drive mechanism being configured to move the movable part reciprocally and linearly back and forth with respect to the device body when operated by the motor; and
the drive mechanism being user-adjustable with the drive mechanism in operational or ready-to-operate condition to vary the obtainable range of travel of the movable part;
the drive mechanism further comprising:
an adjustable support member;
a link member; and
at least one coupling member coupled to the movable part and further coupled to the adjustable support member via the link member;
wherein the adjustable support member is movable between:
a first position in which the movable part reciprocates back and forth in use with a first range of travel;
a second position in which the movable part reciprocates back and forth in use with
a second range of travel; and
adjuster means to move the adjustable support member between the first position and the second position, thereby to adjust the drive mechanism to vary the range of travel.

27. The reciprocator device as claimed in claim 26, wherein:
the coupling member is further coupled to the rotary shaft via the link member.

28. The reciprocator device as claimed in claim 26, wherein:
the coupling member is coupled to the link member, and the link member is coupled to the adjustable support member; and
the link member and the coupling member are further coupled to the rotary shaft.

29. The reciprocator device as claimed in claim 26, wherein:
the coupling member extends between the movable part and the link member; and
the coupling member has a first end which is coupled to the link member at a rotational coupling and a second end which is coupled to the movable part at another rotational coupling.

30. The reciprocator device as claimed in claim 26, wherein the at least one coupling member comprises:
a first coupling member which, via one or more rotational coupling(s), is either or both coupled to and supported upon or by any one or more of: the rotary shaft, the link member, and the adjustable support member; and
a second coupling arranged to extend between the link member and the movable part, the coupling member having a first end which is coupled to the link member at a rotational coupling and a second end which is coupled to the movable part at another rotational coupling.

31. The reciprocator device as claimed in claim 26, wherein:
the link member is supported upon the adjustable support member via a rotational coupling; and
the at least one coupling member comprises:
a first coupling member arranged to extend between the link member and the rotary shaft, the first coupling member having a first end which is coupled to the rotary shaft at a first coupling and a second end which is coupled to the link member at a second, rotational coupling; and
a second coupling member arranged to extend between the link member and the movable part, the second coupling member having a first end which is coupled to the link member at a fourth, rotational coupling and a second end which is coupled to the movable part at a fifth, rotational coupling.

32. The reciprocator device as claimed in claim 26, wherein the first and second positions of the support member are first and second end locations, and the adjuster means operable to move the support member to any suitable position between the end locations, while operating the drive mechanism and whilst reciprocating movement of the movable part is taking place.

33. The reciprocator device as claimed in claim 26, wherein the adjuster means is coupled to the support member through a gear arrangement or a screw arrangement operable to urge the adjustable support member from one location to another relative.

34. The reciprocator device as claimed in claim 26, further comprising control means for operating either or both the adjuster means and the drive mechanism, the control means being operable by a user by means of one or more user-operable control devices.

35. The reciprocator device as claimed in claim 26, wherein the movable part is a linear drive part arranged to be movable linearly back and forth.

36. The reciprocator device as claimed in claim 26, wherein the movable part comprises at least one element or head to interact with the human or animal body, in use, the element or head being any one or more of: a stimulator structure; a massage head; a push/pull head; a cup structure.

37. The reciprocator device as claimed in claim 36, wherein the element or head is detachable.

38. The reciprocator device as claimed in claim 26, being a hand-held device, wherein the device body comprises a hand-held casing in which the drive mechanism is housed.

39. A reciprocator device for acting upon a region or part of a human or animal body for producing at least one effect thereupon, the device comprising:
a device body;
an adjustable drive mechanism, the drive mechanism comprising:
a rotary motor for operating the drive mechanism; and
a rotary member arranged to be driven and rotated by the motor;
a movable part coupled to the drive mechanism;
the drive mechanism being configured to move the movable part reciprocally and linearly back and forth with respect to the device body when operated by the motor; and
the drive mechanism being user-adjustable with the drive mechanism in operational or ready-to-operate condition to vary the obtainable range of travel of the movable part;
the drive mechanism further comprising:
a linkage for linking the movable part to the rotary member; and
an adjustable member coupled to the linkage;
wherein the adjustable member is movable between:
a first setting in which the movable part reciprocates back and forth in use with a first range of travel;
a second setting in which the movable part reciprocates back and forth in use with a second range of travel; and
adjuster means to move the adjustable member between the first setting and the second setting, thereby to adjust the drive mechanism to vary the range of travel.

40. The reciprocator device as claimed in claim 39, wherein the linkage comprises at least one coupling member through which the movable part is coupled to the rotary member.

41. The reciprocator device as claimed in claim 39, wherein the linkage comprises:
at least one coupling member and a link member through which the movable part is coupled to the rotary member, wherein a coupling member is arranged to couple between the movable member and the link member, and the link member is arranged to couple between the coupling member and the adjustable member.

42. The reciprocator as claimed in claim 39, wherein the linkage further comprises:
a link member;
a first coupling member arranged to extend between the link member and the rotary member, the first coupling member having a first end which is coupled to the rotary member at a first coupling and a second end which is coupled to the link member at a second, rotational coupling;
a second coupling member arranged to extend between the link member and the movable part, the second coupling member having a first end which is coupled to the link member at a rotational coupling and a second end which is coupled to the movable part at a rotational coupling.

43. A reciprocator device for acting upon a region or part of a human or animal body for producing at least one effect thereupon, the device comprising:
a device body;
an adjustable drive mechanism, the drive mechanism comprising:
a rotary motor for operating the drive mechanism; and
a rotary shaft arranged to be driven and rotated by the motor;
a movable part coupled to the drive mechanism;
the drive mechanism being configured to move the movable part reciprocally and linearly back and forth with respect to the device body when operated by the motor; and
the drive mechanism being user-adjustable with the drive mechanism in operational or ready-to-operate condition to vary the obtainable range of travel of the movable part;
the drive mechanism further comprising:
an adjustable support member;
wherein the adjustable support member is movable between:
a first position in which the movable part reciprocates back and forth in use with a first range of travel;
a second position in which the movable part reciprocates back and forth in use with a second range of travel; and
adjuster means to move the adjustable support member between the first position and the second position, thereby to adjust the drive mechanism to vary the range of travel.

44. A massager comprising a reciprocator device as claimed in claim 26, the massager having a massage head coupled to the movable part.

45. A reciprocator device for acting upon a region or part of a human or animal body for producing at least one effect thereupon, the reciprocator device comprising:
a device body;
a structure and sealing means for sealably enclosing a part or region of skin of the human or animal body;
a movable part which is arranged to extend from a section of the device body, the movable part being reciprocally movable to travel back and forth with respect to the device body;
the movable part for exerting a force upon the enclosed body part or region of skin of the human or animal body, when the structure in use is sealably located thereupon, in dependence upon the reciprocating movement back and forth; and
means to produce the reciprocating movement of the movable part back and forth with respect to the device body;
wherein the structure comprises:
an outer portion for enclosing the external part or region of skin and the movable part comprising an inner portion arranged inside the outer portion, the inner portion being movable reciprocally back and forth relative to the outer portion, the inner portion being arranged to act upon the enclosed external part or enclosed region of skin;
a flexible diaphragm for coupling the outer portion to the inner portion to permit relative movement between the inner and outer portion;
wherein the inner portion is an end portion of the movable part;
wherein the outer portion comprises any of a ring portion; a bell or cone structure; and a cup structure.

46. The reciprocator device as claimed in claim 45, wherein the outer portion of the structure includes sealing means for facilitating sealably coupling the outer portion of the structure to or locating the outer portion of the structure sealably against the human or animal body, for preventing or restricting air ingress into a region between the movable part and the enclosed body part or body region.

47. The reciprocator device as claimed in claim 45, wherein the structure is arranged such that through placement against the skin, the inner portion including the end portion of the movable part locks in use to the enclosed part or enclosed region of skin through a vacuum lock effect and with the outer portion of the structure urged against the skin, the inner, end portion remains locked to the skin in use throughout reciprocation back and forth.

48. The reciprocator device as claimed in claim 47, wherein the structure comprises an escape passageway through a wall of the outer portion of the structure for letting out fluid or air of the region between the movable part and the sealably enclosed body part or region of skin.

49. The reciprocator device as claimed in claim 48, wherein the escape passageway is configured to be coupled to a pump device for extracting the air from the region between the movable part and the sealably enclosed body part or region of skin.

50. The reciprocator device as claimed in claim 47, wherein the inner portion including the end portion of the movable part is operable for exerting the push/pull force upon the enclosed part or enclosed region of skin for pushing/pulling on the skin back and forth in accordance with the reciprocating movement back and forth and with the end portion remaining locked to the skin throughout.

51. A reciprocator device for acting upon a region or part of a human or animal body for producing at least one effect thereupon, the reciprocator device comprising:
a device body;
a structure and sealing means for sealably enclosing a part or region of skin of the human or animal body;
a movable part which is arranged to extend from a section of the device body, the movable part being reciprocally movable to travel back and forth with respect to the device body;
the movable part for exerting a force upon the enclosed body part or region of skin of the human or animal body, when the structure in use is sealably located thereupon, in dependence upon the reciprocating movement back and forth;
means to produce the reciprocating movement of the movable part back and forth with respect to the device body; and
wherein through the sealed placement of the structure on the body part or region of skin, the movable part lockably couples in use to the enclosed part or enclosed region of skin through a vacuum lock effect and remains locked to the skin in use throughout the reciprocation back and forth.

52. A reciprocator device for acting upon a region or part of a human or animal body for producing at least one effect thereupon, the reciprocator device comprising:
a device body;
a structure and sealing means for sealably enclosing a part or region of skin of the human or animal body, the structure being a push/pull head;
a movable part which is arranged to extend from a section of the device body, the movable part being reciprocally movable to travel back and forth with respect to the device body;
the movable part for exerting a push/pull force upon the enclosed body part or region of skin of the human or animal body, when the structure in use is sealably located thereupon, in dependence upon the reciprocating movement back and forth with the end portion remaining locked to the skin throughout; and
means to produce the reciprocating movement of the movable part back and forth with respect to the device body.

53. The reciprocator device as claimed in claim 52, wherein the movable part lockably couples in use to the enclosed part or enclosed region of skin through a vacuum lock effect.
